(19) United States
(12) Patent Application Publication
(10) Pub. No.: US 2025/0366790 A1
(43) Pub. Date: Dec. 4, 2025

(54) DEVICE AND METHOD FOR EVALUATING QUALITY OF BIOSIGNAL

(71) Applicant: ELECTRONICS AND TELECOMMUNICATIONS RESEARCH INSTITUTE, Daejeon (KR)

(72) Inventors: Min Seong KIM, Daejeon (KR); Hyeon Jeong LEE, Daejeon (KR)

(21) Appl. No.: 18/964,153

(22) Filed: Nov. 29, 2024

(30) Foreign Application Priority Data
May 29, 2024 (KR) 10-2024-0070258

(51) Int. Cl. A61B 5/00

(52) U.S. Cl. CPC A61B 5/7221; A61B 5/7257; A61B 5/7264

(57) ABSTRACT

Provided are a device and method for evaluating quality of a biosignal. The device includes a processor and a memory which stores instructions executed by the processor. The processor normalizes a biosignal and converts the normalized biosignal into a frequency domain, extracts a morphological feature of a spectrum in the frequency domain, and evaluates quality of the biosignal on the basis of the morphological feature.

### CROSS-REFERENCE TO RELATED APPLICATION

This application claims priority to and the benefit of Korean Patent Application No. 10-2024-0070258, filed on May 29, 2024, the disclosure of which is incorporated herein by reference in its entirety.

### BACKGROUND

### 1. Field of the Invention

The present invention relates to a biosignal quality evaluation device and method that normalize a biosignal and convert the normalized biosignal into the frequency domain and extract morphological features of the spectrum in the frequency domain to evaluate the quality of the biosignal.

### 2. Description of Related Art

Biosignals are utilized in many fields, including medicine, healthcare, rehabilitation, and the like, and are used as indicators of the body's response.

Biosignals are signals for measuring minute changes and thus are frequently contaminated by noise such as motion, power noise, ambient light interference, and the like. Accordingly, methods for removing noise from biosignals are being studied, and filtering techniques are being developed to minimize noise. When biosignals are analyzed in the frequency domain, each of the biosignals has its own unique/dominant frequency region, and the region outside the corresponding frequency region is defined as noise. A filtering method for attenuating only such a noise region is being applied. Frequencies generated by body movements of breathing make modifications in a low frequency region, resulting in baseline fluctuation noise of a biosignal, which may be removed using a high-pass filter. Also, noise including electromagnetic interference, power noise, and the like makes modifications in a high-frequency region or a specific frequency region, which may be removed using a low-pass filter, a notch filter, and the like.

Despite these denoising methods being actively studied, a signal-to-noise ratio (SNR) which is widely used in the fields of telecommunications and electronics is utilized as a method for evaluating the quality of raw biosignal data. However, a method of calculating an SNR by calculating an alternating current (AC)/direct current (DC) ratio or the like is inappropriate to apply in an actual measurement environment because it is difficult to define AC and DC components in consideration of the characteristics of biosignals.

The background art of the present invention is disclosed in Korean Patent Publication No. 10-2021-0067760 (Jun. 8, 2021).

### SUMMARY OF THE INVENTION

The present invention is directed to providing a biosignal quality evaluation device and method that normalize a biosignal and convert the normalized biosignal into the frequency domain, extract morphological features of the spectrum in the frequency domain to evaluate the quality of the biosignal, and thus are applicable to various application fields employing biosignals.

According to an aspect of the present invention, there is provided a device for evaluating quality of a biosignal, the device including a processor and a memory configured to store instructions executed by the processor. The processor normalizes biosignals and converts the normalized biosignals into a frequency domain, extracts a morphological feature of a spectrum in the frequency domain, and evaluates quality of the biosignals on the basis of the morphological feature.

The biosignals may correspond to at least one of an electrocardiogram (ECG), a photoplethysmogram (PPG), a ballistocardiogram (BCG), an electromyogram (EMG), an impedance plethysmogram (IPG), a pressure wave, a video plethysmogram (VPG), electrodermal activity (EDA), a galvanic skin response (GSR), an electroencephalogram (EEG), an electrocorticogram (ECoG), and a magnetoencephalogram (MEG).

The morphological feature may be at least one of a maximum, a minimum, a mean, a median, skewness, kurtosis, a peak interval, a ratio between frequency bands, and a ratio of target frequency spectral power to total spectral power in the spectrum graph.

The processor may divide the spectrum into a plurality of regions by frequency band and extract the morphological feature from each of the regions.

The processor may divide the regions according to types of biosignals.

The regions may include a frequency region of interest corresponding to a frequency band of a main signal of the biosignals, a low-frequency noise region including low-frequency noise signal components, and a high-frequency noise region including high-frequency noise signal components.

The processor may divide the regions according to characteristics of the biosignals.

The processor may perform binary or multiclass classification on the quality of the biosignals on the basis of the morphological feature extracted from each of the regions.

The processor may quantify the quality of the biosignals on the basis of the morphological feature extracted from each of the regions.

According to another aspect of the present invention, there is provided a method of evaluating quality of a biosignal, the method including normalizing, by a processor, biosignals, converting, by the processor, the biosignals into a frequency domain, extracting, by the processor, a morphological feature of a spectrum in the frequency domain, and evaluating, by the processor, quality of the biosignals on the basis of the morphological feature.

The biosignals may correspond to at least one of an ECG, a PPG, a BCG, an EMG, an IPG, a pressure wave, a VPG, EDA, a GSR, an EEG, an ECOG, and an MEG.

The morphological feature may be at least one of a maximum, a minimum, a mean, a median, skewness, kurtosis, a peak interval, a ratio between frequency bands, and a ratio of target frequency spectral power to total spectral power in the spectrum graph.

The extracting of the morphological feature may include dividing, by the processor, the spectrum into a plurality of regions by frequency band and extracting the morphological feature from each of the regions.

The extracting of the morphological feature may include dividing, by the processor, the regions according to types of biosignals.

The regions may include a frequency region of interest corresponding to a frequency band of a main signal of the biosignals, a low-frequency noise region including low-frequency noise signal components, and a high-frequency noise region including high-frequency noise signal components.

The extracting of the morphological feature may include dividing, by the processor, the regions according to characteristics of the biosignals.

The evaluating of the quality of the biosignals may include performing, by the processor, binary or multiclass classification on the quality of the biosignals on the basis of the morphological feature extracted from each of the regions.

The evaluating of the quality of the biosignals may include quantifying, by the processor, the quality of the biosignals on the basis of the morphological feature extracted from each of the regions.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of a device for evaluating quality of a biosignal according to an exemplary embodiment of the present invention;

FIGS. 2A and 2B are a set of diagrams of an implementation example of biosignal quality evaluation according to an exemplary embodiment of the present invention;

FIGS. 3A and 3B are a set of diagrams of an implementation example of biosignal quality evaluation according to an exemplary embodiment of the present invention; and

FIG. 4 is a flowchart illustrating a method of evaluating quality of a biosignal according to an exemplary embodiment of the present invention.

### DETAILED DESCRIPTION OF EXEMPLARY EMBODIMENTS

A device and method for evaluating quality of a biosignal according to exemplary embodiments of the present invention will be described below. In this process, the thicknesses of lines, the sizes of components, and the like shown in the drawings may be exaggerated for the purpose of clarity and convenience of description. Also, terms to be described below are defined in consideration of functions in the present invention, and the terms may vary depending on the intention of a user or operator or precedents. Therefore, these terms are to be defined on the basis of the overall content of the specification.

The present invention may be implemented in various different forms and is not limited to embodiments described herein. In the drawings, elements irrelevant to description will be omitted to clearly describe the present invention, and throughout the specification, like reference numerals refer to like elements.

In the specification, when a part is referred to as “including” a certain component, it means that the part may further include other components rather than excluding other components unless otherwise stated.

Description of this specification may be implemented using, for example, a method or process, a device, a software program, a data stream, or a signal. Even if a feature is discussed only in a single form of implementation (e.g., discussed only as a method), the discussed feature may be implemented in another form (e.g., a device or program). The device may be implemented as appropriate hardware, software, firmware, and the like. The method may be implemented in a device such as a processor which generally refers to a processing device including a computer, a microprocessor, an integrated circuit, a programmable logic device, or the like.

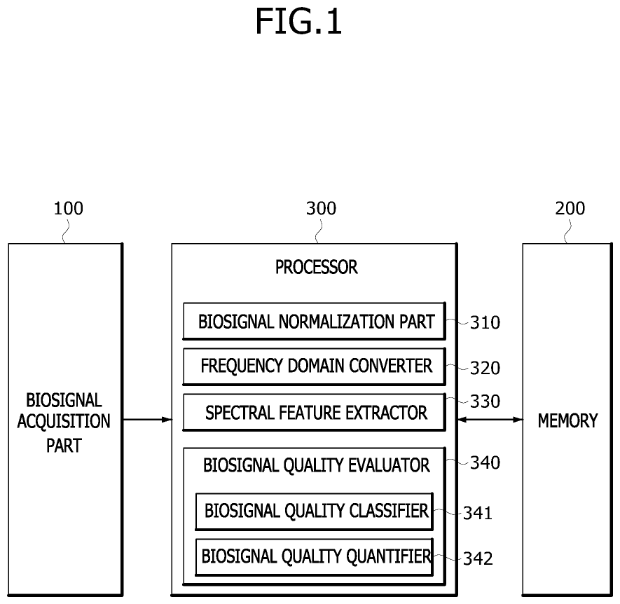

FIG. 1 is a block diagram of a device for evaluating quality of a biosignal according to an exemplary embodiment of the present invention.

Referring to FIG. 1, the device for evaluating quality of a biosignal according to an exemplary embodiment of the present invention may include a biosignal acquisition part 100, a memory 200, and a processor 300.

The biosignal acquisition part 100 may acquire biosignals.

The biosignals may correspond to an electrocardiogram (ECG), a photoplethysmogram (PPG), a ballistocardiogram (BCG), an electromyogram (EMG), an impedance plethysmogram (IPG), a pressure wave, a video plethysmogram (VPG), electrodermal activity (EDA), a galvanic skin response (GSR), an electroencephalogram (EEG), an electrocorticogram (ECoG), and a magnetoencephalogram (MEG). There is no particular limitation on the types of biosignals.

The biosignal acquisition part 100 may acquire biosignals directly from a biosignal measurement device (not shown).

The biosignal measurement device may be a patient monitor (PM), a wearable device, and a biosignal meter.

The biosignal acquisition part 100 may acquire biosignals in real time from a subject via the biosignal measurement device.

The biosignal acquisition part 100 may acquire biosignals from a data file in which biosignals are stored.

The memory 200 may store various data used by the processor 300. Instructions for performing operations, steps, or the like according to exemplary embodiments of the present invention may be stored as data. In other words, the memory 200 may store instructions for evaluating the quality of biosignals.

The memory 200 may include at least one storage medium among a flash memory, a hard disk, a multimedia card micro-type memory, a card-type memory, a random access memory (RAM), a static random access memory (SRAM), a read-only memory (ROM), a programmable read-only memory (PROM), an erasable programmable read-only memory (EPROM), and an electrically erasable programmable read-only memory (EEPROM).

The processor 300 may be connected to the memory 200 and execute instructions stored in the memory 200. The processor 300 may execute instructions stored in the memory 200 to control at least one other component (e.g., a hardware or software component) connected to the processor 300 and perform various data processing and calculations.

Also, the processor 300 may be configured such that elements for performing functions are separated at the hardware, software, or logic level. In this case, dedicated hardware for performing each function may be used. To this end, the processor 300 may be implemented as at least one of an application specific integrated circuit (ASIC), a digital signal processor (DSP), programmable logic devices (PLDs), field programmable gate arrays (FPGAs), a central processing unit (CPU), a microcontroller, and/or microprocessors or include at least one thereof.

The processor 300 may be implemented as a CPU or a system on chip (SoC). The processor 300 may run an operating system (OS) or application to control a plurality of hardware or software components connected thereto and perform various data processing and calculations. The processor 300 may be configured to execute at least one instruction stored in the memory 200 and store the execution result data.

The processor 300 may normalize a biosignal and convert the normalized biosignal into the frequency domain, extract a morphological feature of the spectrum in the frequency domain, and then evaluate the quality of the biosignal on the basis of the extracted morphological feature.

The processor 300 may include a biosignal normalization part 310, a frequency domain converter 320, a spectral feature extractor 330, and a biosignal quality evaluator 340.

The biosignal normalization part 310 may normalize the amplitude of each biosignal to a value within a set range.

The scale of a normalized result value may have a range of [0, 1], [−1, 1], or the like.

As a normalization technique, min-max normalization or max-abs normalization may be applied, but there is no particular limitation on the normalization technique.

The frequency domain converter 320 may convert the biosignal normalized by the biosignal normalization part 310 into the frequency domain.

As a technique for converting the normalized biosignal into the frequency domain, a Fourier transform or Laplace transform may be applied, but there is no particular limitation on the conversion technique.

The biosignal, which is in the time-series domain, may be changed to a signal in the frequency domain and represented in the form of a spectrum and represented as the calculated power of each frequency.

The spectral feature extractor 330 may extract a morphological feature of the spectrum in the frequency domain.

The morphological feature of the spectrum may be a maximum, a minimum, a mean, a median, skewness, kurtosis, a peak interval, a ratio between frequency bands, and a ratio of target frequency spectral power to total spectral power, but there is no particular limitation on the morphological feature in the spectrum graph.

The spectral feature extractor 330 may divide the spectrum into a plurality of regions according to frequency bands.

The spectral feature extractor 330 may extract a morphological feature from each divided region.

The spectral feature extractor 330 may diversely divide the spectrum into a plurality of regions depending on the type of biosignal. For example, the spectral feature extractor 330 may divide regions differently depending on an ECG, a PPG, a BCG, an EMG, an IPG, a pressure wave, and a VPG.

Regions may be divided as a frequency region of interest corresponding to a frequency band of a main signal among biosignals, a low-frequency noise region including low-frequency noise signal components, and a high-frequency noise region including high-frequency noise signal components.

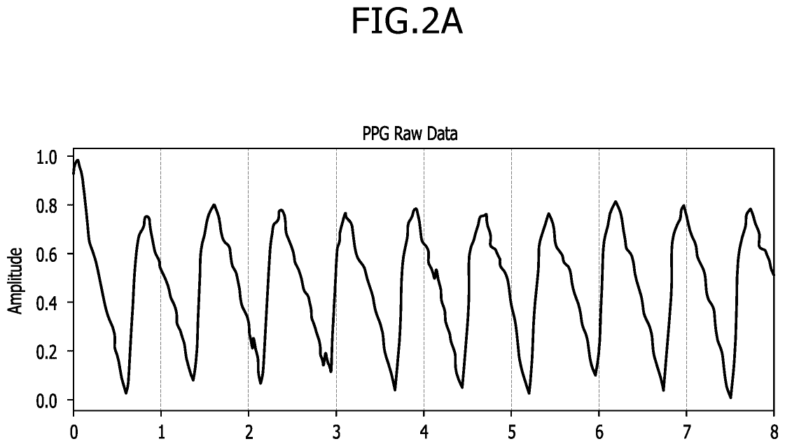

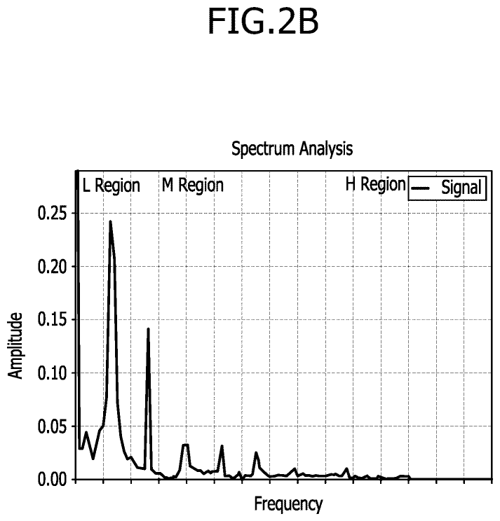

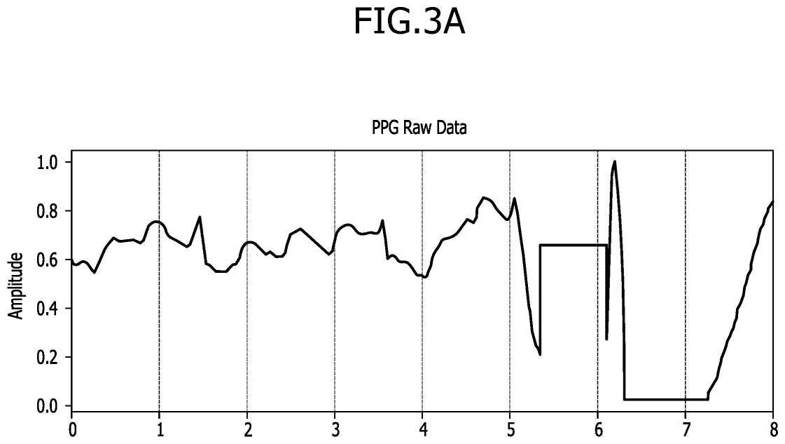

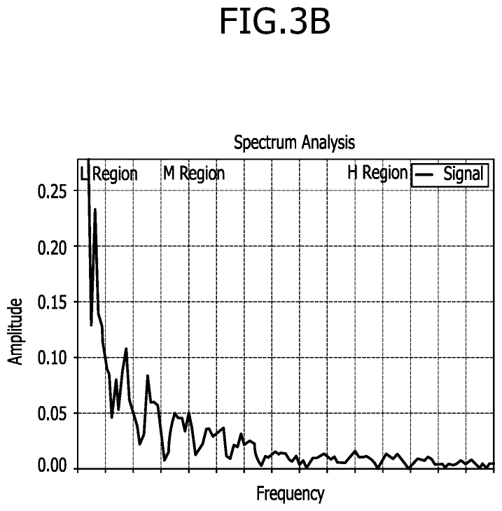

FIG. 2 is a set of diagrams of an implementation example of biosignal quality evaluation according to an exemplary embodiment of the present invention, and FIG. 3 is a set of diagrams of an implementation example of biosignal quality evaluation according to an exemplary embodiment of the present invention.

Referring to FIGS. 2 and 3, an example is shown in which a PPG spectrum is divided into a frequency region of interest (M region), a low-frequency noise region (L region), and a high-frequency noise region (H region).

The spectral feature extractor 330 may extract a morphological feature through morphological analysis of the spectrum of main signal-related components in the frequency region of interest.

The spectral feature extractor 330 may extract a morphological feature through morphological analysis of the spectrum of low-frequency noise signal components in the low-frequency noise region.

The spectral feature extractor 330 may extract a morphological feature through morphological analysis of the spectrum of high-frequency noise signal components in the high-frequency noise region.

The spectral feature extractor 330 may also divide regions according to characteristics of the biosignal.

In other words, the spectral feature extractor 330 may set the frequency range of each region in consideration of universal characteristics or individual characteristics of the biosignals.

For example, when the frequency range of each region is set in consideration of individual characteristics of biosignals, the spectral feature extractor 330 may calculate a heart rate on the basis of peak points detected in a PPG or ECG and utilize the heart rate to set the frequency region of interest, the low-frequency noise region, and the high-frequency noise region in real time or variably.

The biosignal quality evaluator 340 may evaluate the quality of the biosignal on the basis of region-specific morphological features.

The biosignal quality evaluator 340 may include a biosignal quality classifier 341 and a biosignal quality quantifier 342.

The biosignal quality classifier 341 may perform binary classification (good or bad) on the biosignal on the basis of the region-specific morphological features.

The biosignal quality classifier 341 may perform multiclass classification (high quality, acceptable, low quality, not acceptable) on the biosignal on the basis of the region-specific morphological features.

For example, the biosignal quality classifier 341 may compare the morphological features extracted from the frequency region of interest, the low-frequency noise region, and the high-frequency noise region with threshold values separately set for the regions and classify the quality of the biosignal according to the comparison result.

The biosignal quality quantifier 342 may quantify the quality of the biosignal on the basis of the region-specific morphological features.

The biosignal quality quantifier 342 may combine at least one morphological feature of each of the frequency region of interest, the low-frequency noise region, and the high-frequency noise region and calculate the sum, ratio, mean, and the like of the morphological features.

The biosignal quality quantifier 342 may quantify the quality of the biosignal as a value within a range of [0, 1], [−1, 1], [−100, 100], or the like on the basis of the calculated sum, ratio, and mean of the morphological features.

In the present embodiment, to facilitate understanding of the embodiment, the biosignal normalization part 310, the frequency domain converter 320, the spectral feature extractor 330, the biosignal quality classifier 341, and the biosignal quality evaluator 340 are described as separate elements in the processor 300. However, according to an embodiment, the processor 300 may be implemented as an element that integrally runs the sub-elements.

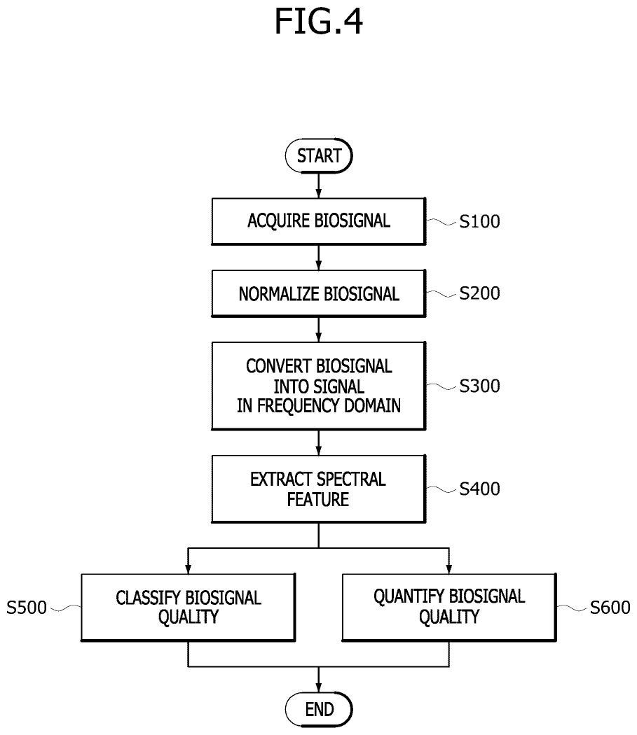

A method of evaluating quality of a biosignal according to an exemplary embodiment of the present invention will be described below with reference to FIG. 4.

FIG. 4 is a flowchart illustrating a method of evaluating quality of a biosignal according to an exemplary embodiment of the present invention.

Referring to FIG. 4, the biosignal acquisition part 100 may acquire biosignals (S100).

The biosignal acquisition part 100 may acquire biosignals directly from a biosignal meter, acquire biosignals in real time from a subject via a biosignal meter, or acquire biosignals from a data file.

The biosignals may be at least one of an ECG, a PPG, a BCG, an EMG, an IPG, a pressure wave, a VPG, EDA, a GSR, an EEG, an ECOG, and an MEG.

The processor 300 may normalize the amplitudes of the biosignals to values within a set range by applying min-max normalization or max-abs normalization (S200). The scale of normalized result values may have a range of [0, 1], [−1, 1], or the like.

The processor 300 may convert the normalized biosignals into signals in the frequency domain by applying a Fourier transform or Laplace transform (S300).

The processor 300 may extract a morphological feature of a spectrum in the converted frequency domain (S400).

The morphological feature of the spectrum may be a maximum, a minimum, a mean, a median, skewness, kurtosis, a peak interval, a ratio between frequency bands, and a ratio of target frequency spectral power to total spectral power in the spectrum graph.

The processor 300 may divide the spectrum into a plurality of regions according to frequency band and extract a morphological feature from each of the divided regions.

The regions may be classified as a frequency region of interest corresponding to a frequency band of a main signal of the biosignals, a low-frequency noise region including low-frequency noise signal components, and a high-frequency noise region including high-frequency noise signal components.

Meanwhile, the processor 300 may divide the regions according to characteristics of the biosignals. In this case, the processor 300 may set the frequency range of each region in consideration of universal characteristics or individual characteristics of the biosignals.

The processor 300 may perform binary classification (good or bad) on the biosignals on the basis of the region-specific morphological features or perform multiclass classification (high quality, acceptable, low quality, not acceptable) on the biosignals on the basis of the region-specific morphological features (S500). The processor 300 may compare the morphological features extracted from the frequency region of interest, the low-frequency noise region, and the high-frequency noise region with threshold values separately set for the regions and classify the quality of the biosignals based on the comparison results.

Also, the processor 300 may quantify the quality of the biosignals on the basis of the sum, ratio, and mean of morphological features obtained by combining at least one morphological feature of each region (S600).

As described above, a device and method for evaluating quality of a biosignal according to an exemplary embodiment of the present invention can determine and quantify the quality of raw biosignal data.

Also, a device and method for evaluating quality of a biosignal according to an exemplary embodiment of the present invention can be utilized as a preprocessing technology in various application fields employing biosignals and allow a medical and healthcare system to provide an accurate service to a user on the basis of the preprocessing technology.

In addition, a device and method for evaluating quality of a biosignal according to an exemplary embodiment of the present invention allow a task to be performed on the basis of biosignal data and allow evaluation results to be utilized as a reliability indicator for results of performing the task.

A device and method for evaluating quality of a biosignal according to an aspect of the present invention can determine and quantify the quality of raw biosignal data.

A device and method for evaluating quality of a biosignal according to another aspect of the present invention can be utilized as a preprocessing technology in various application fields employing biosignals and allow a medical and healthcare system to provide an accurate service to a user on the basis of the preprocessing technology.

A device and method for evaluating quality of a biosignal according to another aspect of the present invention allow a task to be performed on the basis of biosignal data and allow evaluation results to be utilized as a reliability indicator for results of performing the task.

Although the present invention has been described above with reference to embodiments illustrated in the drawings, the embodiments are merely illustrative, and those skilled in the art should understand that various modifications and other equivalent embodiments can be made from the embodiments. Therefore, the technical scope of the present invention should be determined from the following claims.

What is claimed is:

1. A device for evaluating quality of a biosignal, the device comprising:
a processor; and
a memory configured to store instructions executed by the processor,
wherein the processor normalizes biosignals and converts the normalized biosignals into a frequency domain, extracts a morphological feature of a spectrum in the frequency domain, and evaluates quality of the biosignals on the basis of the morphological feature.

2. The device of claim 1, wherein the biosignals correspond to at least one of an electrocardiogram (ECG), a photoplethysmogram (PPG), a ballistocardiogram (BCG), an electromyogram (EMG), an impedance plethysmogram (IPG), a pressure wave, a video plethysmogram (VPG), electrodermal activity (EDA), a galvanic skin response (GSR), an electroencephalogram (EEG), an electrocorticogram (ECoG), and a magnetoencephalogram (MEG).

3. The device of claim 1, wherein the morphological feature is at least one of a maximum, a minimum, a mean, a median, skewness, kurtosis, a peak interval, a ratio between frequency bands, and a ratio of target frequency spectral power to total spectral power in the spectrum graph.

4. The device of claim 1, wherein the processor divides the spectrum into a plurality of regions by frequency band and extracts the morphological feature from each of the regions.

5. The device of claim 4, wherein the processor divides the regions according to types of biosignals.

6. The device of claim 4, wherein the regions include a frequency region of interest corresponding to a frequency band of a main signal of the biosignals, a low-frequency noise region including low-frequency noise signal components, and a high-frequency noise region including high-frequency noise signal components.

7. The device of claim 4, wherein the processor divides the regions according to characteristics of the biosignals.

8. The device of claim 4, wherein the processor performs binary or multiclass classification on the quality of the biosignals on the basis of the morphological feature extracted from each of the regions.

9. The device of claim 4, wherein the processor quantifies the quality of the biosignals on the basis of the morphological feature extracted from each of the regions.

10. A method of evaluating quality of a biosignal, the method comprising:
normalizing, by a processor, biosignals;
converting, by the processor, the biosignals into a frequency domain;
extracting, by the processor, a morphological feature of a spectrum in the frequency domain; and
evaluating, by the processor, quality of the biosignals on the basis of the morphological feature.

11. The method of claim 10, wherein the biosignals correspond to at least one of an electrocardiogram (ECG), a photoplethysmogram (PPG), a ballistocardiogram (BCG), an electromyogram (EMG), an impedance plethysmogram (IPG), a pressure wave, a video plethysmogram (VPG), electrodermal activity (EDA), a galvanic skin response (GSR), an electroencephalogram (EEG), an electrocorticogram (ECoG), and a magnetoencephalogram (MEG).

12. The method of claim 10, wherein the morphological feature is at least one of a maximum, a minimum, a mean, a median, skewness, kurtosis, a peak interval, a ratio between frequency bands, and a ratio of target frequency spectral power to total spectral power in the spectrum graph.

13. The method of claim 10, wherein the extracting of the morphological feature comprises dividing, by the processor, the spectrum into a plurality of regions by frequency band and extracting the morphological feature from each of the regions.

14. The method of claim 13, wherein the extracting of the morphological feature comprises dividing, by the processor, the regions according to types of biosignals.

15. The method of claim 13, wherein the regions include a frequency region of interest corresponding to a frequency band of a main signal of the biosignals, a low-frequency noise region including low-frequency noise signal components, and a high-frequency noise region including high-frequency noise signal components.

16. The device of claim 13, wherein the extracting of the morphological feature comprises dividing, by the processor, the regions according to characteristics of the biosignals.

17. The device of claim 13, wherein the evaluating of the quality of the biosignals comprises performing, by the processor, binary or multiclass classification on the quality of the biosignals on the basis of the morphological feature extracted from each of the regions.

18. The device of claim 13, wherein the evaluating of the quality of the biosignals comprises quantifying, by the processor, the quality of the biosignals on the basis of the morphological feature extracted from each of the regions.
